(19) United States
(12) Patent Application Publication
(10) Pub. No.: US 2022/0101964 A1
(43) Pub. Date: Mar. 31, 2022

(54) MEDICAL DATA MANAGEMENT SYSTEM

(71) Applicant: Siemens Healthcare GmbH, Erlangen (DE)

(72) Inventors: Srikrishna PRASAD, Erlangen (DE); Michael KELM, Erlangen (DE); Ute ROSENBAUM, Kempten (DE)

(73) Assignee: Siemens Healthcare GmbH, Erlangen (DE)

(21) Appl. No.: 17/476,526

(22) Filed: Sep. 16, 2021

(30) Foreign Application Priority Data
Sep. 28, 2020 (DE) 10 2020 212 187.7

(51) Int. Cl. G16H 10/60; G16H 40/20; G06F 21/62; G06F 16/245

(52) U.S. Cl. CPC G16H 10/60; G16H 40/20; G16H 30/20; G06F 16/245; G06F 21/6254

(57) ABSTRACT

A medical data management system is for managing medical data. In an embodiment, the system includes a medical data gateway, including a processor connectable to a plurality of input devices, the medical data gateway being connected to a local network. Further, the processor is configured to carry out: collecting medical data, associated to a non-pseudonymized patient identifier and a data source identifier, from an input device; pseudonymizing at least the non-pseudonymized patient identifier of the medical data; exporting the pseudonymized medical data to a remote storage, the remote storage being part of a remote network external to the local network; allowing access to the non-pseudonymized patient identifier to a local application, running in the local network, and/or refusing access to the non-pseudonymized patient identifier to a remote application, running outside of the local network. A corresponding medical data management method is for managing medical data.

### PRIORITY STATEMENT

The present application hereby claims priority under 35 U.S.C. § 119 to German patent application number DE102020212187.7 filed Sep. 28, 2020, the entire contents of which are hereby incorporated herein by reference.

### FIELD

Example embodiments of the invention generally relate to a medical data management system.

### BACKGROUND

Modern medical equipment is often capable of producing output in the form of digital data. Examples thereof can be, for instance, images from a magnetic resonance scanner or from an X-ray scanner, electrocardiogram and blood pressure data, temperature of a patient, etc. With the increase in digitalization, the number of medical devices capable of outputting data in digital form is ever increasing.

The outputted data can have a variety of formats, due to the fact that the data can represent different physical values and to the fact that the various devices can be made by different manufacturers with different approaches.

The outputted data can further have a variety of use and users. For instance, images from a magnetic resonance scanner can be used by a doctor for diagnosing a patient. In this case, the data can be visualized on a monitor connected directly to the scanner, or transmitted to a PC in the doctor's office. Still for instance, data can be transferred to medical insurances for evaluating claims. As another example, data can be made available to companies involved in clinical trials. Still further, service providers might be allowed access to the medical data for various operations, such as artificial intelligence analysis of the data.

Different users can further have different rights. For instance, a doctor and an insurance company might need to access the medical data, such as an X-ray image, and the patient data associated to it, such as sex, age, name, and address. A company involved in a clinical trial carried out at a hospital might on the other hand only be allowed access to the medical data, without any access to the patient data, both for privacy reasons and for compliancy with protocols such as double-blind clinical trials. Still further, a service provider performing processing of the data by artificial intelligence is usually excluded from accessing the patient's identification data.

Moreover, the data can be made accessible within an hospital network as well as outside of it, for instance through a cloud storage or more generally an external network, so as to be accessible for instance to insurance companies, companies involved with clinical trials, governments, external service providers, etc.

It is thus becoming increasingly complex to efficiently manage the medical data and its access by various users. Solutions currently existing on the market, such as the teamplay Digital Health Cloud Platform from Siemens support data push/pull from hospital systems to cloud with strict compliance to HIPAA/GDPR regulations for both data in rest and motion either inside hospital network or outside network.

A problem with such a stringent security measure is that the data is either pseudonymized or encrypted, on a regular basis, even if data is accessed inside hospital network, for instance by a doctor carrying out a diagnosis. This hinders management of workflow as patient data cannot be easily re-identified, after having been pseudonymized or encrypted, and hence data sets cannot be easily associated to a specific patient.

Current solutions thus mainly focus on usage of pseudonymized or encrypted data both at rest and in motion and there is no re-identification mechanism in the application to address above issue. Some re-identification mechanisms only re-identify one data set, from a specific type of data related to a specific output from a given device. However, as indicated above, multitude of datasets can be associated with a patient inside a hospital during treatment. It thus becomes difficult to adopt such mechanisms to address needs of cloud healthcare application's to provision re-identified datasets from various hospital data systems. Also application developer vendor has to invest time and money for developing applications suitable for on-premise environment and cloud environment, which waste of time and money.

### SUMMARY

At least one embodiment of the invention provides a data management system for medical data, which allows data to be efficiently accessed inside a hospital network as well as outside of it, while entrusting various users with various privacy levels in compliance with legal privacy requirements. At least one embodiment of the invention allows such a system to operate on various data sources from various devices.

Generally, the inventors have discovered that the advantages above can be achieved by a system, for example, which implements pseudonymizing of the patient identifier portion of the medical data, prior to render it available to various output devices, or users, while storing the non-pseudonymized patient identifier in association with the pseudonymized patient identifier.

This allows the medical data to be stored and made accessible only with the pseudonymized patient identifier. For those uses in which access is needed to the non-pseudonymized patient identifiers, the system can allow the re-identification to be performed only to those devices and/or users who have right to access this feature.

Thanks to this approach it is advantageously possible to treat all medical data within a consistent framework, by pseudonymizing all incoming data prior to storing it on a storage accessible to all users and/or output devices. This further advantageously allows all users and/or output devices to be designed for accessing the data in a similar manner, independently on the privacy rights associated to the users and/or output devices. On top of this basic functionality, at least one embodiment of the invention allows those users and/or output devices with an increased privacy access level, to request a re-identification of the pseudonymized data so as to additionally get access to the non-pseudonymized patient identifier.

An embodiment of the invention can therefore relate to a medical data management system for managing medical data comprising: a medical data gateway, comprising a processor connectable to a plurality of input devices, wherein the medical data gateway is connected to a local network, the processor being configured to carry out the step of: collecting medical data, associated to a non-pseudonymized patient identifier and a data source identifier, from an input device of the plurality of input devices, pseudonymizing at least the non-pseudonymized patient identifier of the medical data, exporting the pseudonymized medical data to a remote storage, the remote storage being part of a remote network, external to the local network, allowing access to the non-pseudonymized patient identifier to a local application, running in the local network, and/or refusing access to the non-pseudonymized patient identifier to a remote application, running outside of the local network.

A further embodiment of the invention can relate to a medical data management method for managing medical data comprising the steps of: at a medical data gateway connected to a local network, collecting medical data, associated to a non-pseudonymized patient identifier and a data source identifier, from an input device of a plurality of input devices, pseudonymizing at least the non-pseudonymized patient identifier of the medical data, exporting the pseudonymized medical data to a remote storage, the remote storage being part of a remote network, external to the local network, allowing access to the non-pseudonymized patient identifier to a local application, running in the local network, and/or refusing access to the non-pseudonymized patient identifier to a remote application, running outside of the local network.

At least one embodiment is directed to a computer program or a computer-program product or a computer-readable storage medium, including program code. The program code can be loaded and executed by at least one processor. Upon loading and executing the program code, the at least one processor can perform a method for managing medical data. The method includes collecting medical data associated to a non-pseudonymized patient identifier and a data source identifier from an input device of a plurality of input devices. The method also includes pseudonymizing at least the non-pseudonymized patient identifier of the medical data. The method also includes exporting the pseudonymized medical data to a remote storage. The remote storage can be part of a remote network external to the local network. The method also includes allowing access to the non-pseudonymized patient identifier to a local application running on the local network. Alternatively or additionally, the method can include refusing access to the non-pseudonymized patient identifier to a remote application, running outside of the local network.

At least one embodiment is directed to a medical data management system for managing medical data, comprising:

a medical data gateway, comprising at least one processor connectable to a plurality of input devices, the medical data gateway being connected to a local network and the at least one processor being configured to carry out at least:

collecting medical data, associated to a non-pseudonymized patient identifier and a data source identifier, from an input device of the plurality of input devices, pseudonymizing at least the non-pseudonymized patient identifier of the medical data to produce pseudonymized medical data, exporting the pseudonymized medical data to a remote storage, the remote storage being part of a remote network, external to the local network, and at least one of making the non-pseudonymized patient identifier accessible to a local application, running in the local network, and making the non-pseudonymized patient identifier non-accessible to a remote application, running outside of the local network.

At least one embodiment is directed to a medical data management method for managing medical data, comprising:

collecting medical data, at a medical data gateway connected to a local network, associated to a non-pseudonymized patient identifier and a data source identifier, from an input device of a plurality of input devices;

pseudonymizing at least the non-pseudonymized patient identifier of the medical data to create pseudonymized medical data;

exporting the pseudonymized medical data to a remote storage, the remote storage being part of a remote network, external to the local network; and at least one of making the non-pseudonymized patient identifier accessible to a local application, running in the local network, and making the non-pseudonymized patient identifier inaccessible to a remote application, running outside of the local network.

### BRIEF DESCRIPTION OF THE DRAWINGS

FIG. 1 schematically illustrates a medical data management system 1000;

FIGS. 2-5 schematically illustrates possible operations of the medical data management system 1000.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. At least one embodiment of the present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without subdividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Bluray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Bluray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Nonlimiting examples of the non-transitory computer-readable medium include, but are not limited to, rewriteable nonvolatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable nonvolatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

An embodiment of the invention can therefore relate to a medical data management system for managing medical data comprising: a medical data gateway, comprising a processor connectable to a plurality of input devices, wherein the medical data gateway is connected to a local network, the processor being configured to carry out the step of: collecting medical data, associated to a non-pseudonymized patient identifier and a data source identifier, from an input device of the plurality of input devices, pseudonymizing at least the non-pseudonymized patient identifier of the medical data, exporting the pseudonymized medical data to a remote storage, the remote storage being part of a remote network, external to the local network, allowing access to the non-pseudonymized patient identifier to a local application, running in the local network, and/or refusing access to the non-pseudonymized patient identifier to a remote application, running outside of the local network.

Example input devices can include medical imaging devices, e.g., a computed tomography (CT) scanner, a magnetic resonance imaging (MRI) scanner, an x-ray imager, and ultrasound imager. Example input devices could also include, e.g., an electrocardiography device, a blood testing control computer, a blood pressure measurement device, a pulse measurement device, etc.

Pseudonymizing can—according to the techniques described herein—correspond to removing or altering all or at least some of information from medical data that could enable to conclude on the identity of the associated patient. For example, a name of the patient may be removed or altered so as to prevent identification of the patient through the name. For instance, address of living or telephone numbers may be deleted. For example, when pseudonymizing, a hash value may be determined based on the patient identifier. Parts of the patient identifier may also be deleted. It would be possible to determine an encrypted representation of the patient identifier when pseudonymizing the patient identifier.

For example, the degree of pseudonymizing may depend on one or more privacy profiles. For instance, it would be conceivable that—depending on the privacy profile—more or less personal information relating to the patient is removed from the medical data. For instance, the privacy profile may specify certain criteria that can be used by a respective algorithm to selectively discard or convert or otherwise modify privacy-relevant data.

Collecting the medical data can include requesting the medical data from a respective input device. It would also be possible that the medical data is provided in a push communication from the respective input device. For example, a Picture archiving and communication system (PACS) may provide notifications to subscribers.

According to various examples, the medical data may be acquired using an imaging workflow. For instance, a CT scan of a brain tumour patient could benefit from respective CT workflows.

As a general rule, the medical data may include one or more images, e.g., a movie. Images included in the medical data may be provided in the Digital Imaging and Communications in Medicine (DICOM) format.

The data source identifier can be implemented, e.g., by a serial number of the respective input device. It would also be possible that the source identifier is an address of the respective input device in a network connecting the medical data gateway and the various input devices.

A local network may be defined by a trusted domain. Within the trusted domain, only pre-authorized devices may be present. Such devices may be assigned with appropriate credentials, e.g., by a central authority of the local network. The local network may be a secure domain of a PACS. The local network can include archives for PACS data. The local network can communicate with untrusted devices that have not be assigned with credentials of the local network.

Whether a certain application runs in the local network or runs outside of the local network may be dependent on a host device executing the respective application. For instance, it would be conceivable that the local network is an on-premise network of a hospital and an application is executed locally on a device situated in the local network. In some scenarios, it would be conceivable that a host device is off-premise and connected to other devices of the local network via a virtual private network connection established via the Internet. The local network may be characterized by a range of network addresses or a common address space. The local network may be characterized by shared encryption secrets that facilitate communication between devices of the local network. The local network can be connected via a gateway to, e.g., the Internet.

Allowing access to the non-pseudonymized patient identifier can correspond to forwarding the patient identifier upon receiving a respective query.

For example, it would be possible that the processor is configured to either allow access to the non-pseudonymized patient identifier or refuse access to the non-pseudonymized patient identifier, depending on whether a respective query is received from an application that is located in the local network, i.e., a local application, or an application that runs outside of the local network.

Thus, generally speaking, access control to the non-pseudonymized patient identifier is implemented, e.g., depending on a decision criteria as identified above regarding the point of execution of a requesting application. However, it should be understood that, as a general rule, further or other decision criteria could be taken into consideration in addition to the point of execution or alternatively to the point of execution. For instance, decision criteria regarding the selective grant of access can also comprise a temporal aspect, e.g., depending on the time of the day or the day of the week. The decision criterion could also include identities of a requesting subscriber, e.g., irrespective or in addition to the particular application providing the query for the subscriber.

In some embodiments, the medical data gateway can further comprise a storage, wherein, for the step of pseudonymizing, the processor can further be configured to carry out the steps of: creating a pseudonymized patient identifier based on the non-pseudonymized patient identifier and the data source identifier, associating the medical data to the pseudonymized patient identifier, and storing the pseudonymized patient identifier and the non-pseudonymized patient identifier in the storage.

For example, a pointer (also termed association) may be implemented that links the pseudonymized patient identifier with the non-pseudonymized patient identifier. In such a manner, it may be possible to retrieve the non-pseudonymized patient identifier from the storage if in possession of the pseudonymized patient identifier and optionally the association. In some scenarios, to retrieve the non-pseudonymized patient identifier based on the pseudonymized patient identifier, additional credentials may be required, e.g., an authorization code, etc. This may be used to decrypt the association.

In some embodiments, for the step of exporting, the processor can further be configured to carry out the steps of:

transferring the pseudonymized medical data to the remote storage, deleting the pseudonymized medical data from the medical data gateway.

Deleting the pseudonymized medical data can include permanently removing so as to avoid further access to the pseudonymized medical data.

In some embodiments, for the step of accessing, the processor can further be configured to carry out the steps of: receiving a conversion request comprising the pseudonymized patient identifier, evaluating whether the conversion request has sufficient rights for obtaining the non-pseudonymized patient identifier, in case of a positive result of the evaluating step, converting the pseudonymized patient identifier into the corresponding non-pseudonymized patient identifier.

For example, evaluating whether the conversion request has sufficient rights may be based on credentials associated with an originator of the request. For example, it would be possible that credentials are implemented using, e.g., a certifycate that has been issued by a trusted authority. For example, public-private cryptographic keying pairs could be used. For instance, the originator may be a certain type of application or an application being executed on-premise, e.g., in a local network, or off-premise. For instance, applications that are executed in the local network may be considered to have sufficient rights.

By such techniques, it is possible to ensure that unauthorised requests are rejected. Thereby, a high standard of security for the pseudonymized patient identifier can be maintained.

In some embodiments, the processor can further be configured to carry out the step of: receiving a data availability notification, comprising the data source identifier and the patient identifier. For example, the data availability notification may be broadcasted within the local network. It would be possible that data availability notification is pushed to subscribers of a respective notification service. The data availability notification may be generally indicative of new medical data becoming available, in particular, of new medical data becoming available that includes a patient identifier that may be subject to pseudonymization.

In some embodiments, the processor can further be configured to carry out the step of: receiving a data query, comprising the data source identifier and the patient identifier or including a pointer to the data source identifier and the patient identifier. By means of the data query, a push service can be implemented, e.g., in the context of a PACS. Availability of new medical data may be signalled. Thereby, the pseudonomyization can be in response to the medical data becoming available.

In some embodiments, the system can further comprise a multiplexer configured to connect the processor to an input device identified by the data source identifier. The multiplexer may be implemented in hardware and/or software.

In some embodiments, the system can further comprise a demultiplexer configured to connect the processor to an output device.

A further embodiment of the invention can relate to a medical data management method for managing medical data comprising the steps of: at a medical data gateway connected to a local network, collecting medical data, associated to a non-pseudonymized patient identifier and a data source identifier, from an input device of a plurality of input devices, pseudonymizing at least the non-pseudonymized patient identifier of the medical data, exporting the pseudonymized medical data to a remote storage, the remote storage being part of a remote network, external to the local network, allowing access to the non-pseudonymized patient identifier to a local application, running in the local network, and/or refusing access to the non-pseudonymized patient identifier to a remote application, running outside of the local network.

In some embodiments, the step of pseudonymizing can comprise the steps of: creating a pseudonymized patient identifier based on the non-pseudonymized patient identifier and the data source identifier, associating the medical data to the pseudonymized patient identifier, and storing the pseudonymized patient identifier and the non-pseudonymized patient identifier in a storage.

In some embodiments, the step of exporting can comprise the steps of: transferring the pseudonymized medical data to the remote storage, deleting the pseudonymized medical data from the medical data gateway.

In some embodiments, the step of accessing can comprise the steps of: receiving a conversion request comprising the pseudonymized patient identifier, evaluating whether the conversion request has sufficient rights for obtaining the non-pseudonymized patient identifier, in case of a positive result of the evaluating step, converting the pseudonymized patient identifier into the corresponding non-pseudonymized patient identifier.

In some embodiments, the method can further comprise the step of: receiving a data availability notification, comprising the data source identifier and the patient identifier.

In some embodiments, the method can further comprise the step of: receiving a data query, comprising the data source identifier and the patient identifier.

In some embodiments, the method can further comprise the step of: multiplexing a connection between a processor of the medical data gateway and an input device identified by the data source identifier.

In some embodiments, the method can further comprise the step of: demultiplexing a connection between a processor of the medical data gateway and an output device.

A computer program or a computer-program product or a computer-readable storage medium includes program code. The program code can be loaded and executed by at least one processor. Upon loading and executing the program code, the at least one processor can perform a method for managing medical data. The method includes collecting medical data associated to a non-pseudonymized patient identifier and a data source identifier from an input device of a plurality of input devices. The method also includes pseudonymizing at least the non-pseudonymized patient identifier of the medical data. The method also includes exporting the pseudonymized medical data to a remote storage. The remote storage can be part of a remote network external to the local network. The method also includes allowing access to the non-pseudonymized patient identifier to a local application running on the local network. Alternatively or additionally, the method can include refusing access to the non-pseudonymized patient identifier to a remote application, running outside of the local network.

It is to be understood that the features mentioned above and those yet to be explained below may be used not only in the respective combinations indicated, but also in other combinations or in isolation without departing from the scope of the invention.

Some examples of the present disclosure generally provide for a plurality of circuits or other electrical devices. All references to the circuits and other electrical devices and the functionality provided by each are not intended to be limited to encompassing only what is illustrated and described herein. While particular labels may be assigned to the various circuits or other electrical devices disclosed, such labels are not intended to limit the scope of operation for the circuits and the other electrical devices. Such circuits and other electrical devices may be combined with each other and/or separated in any manner based on the particular type of electrical implementation that is desired. It is recognized that any circuit or other electrical device disclosed herein may include any number of microcontrollers, a graphics processor unit (GPU), integrated circuits, memory devices (e.g., FLASH, random access memory (RAM), read only memory (ROM), electrically programmable read only memory (EPROM), electrically erasable programmable read only memory (EEPROM), or other suitable variants thereof), and software which co-act with one another to perform operation(s) disclosed herein. In addition, any one or more of the electrical devices may be configured to execute a program code that is embodied in a non-transitory computer readable medium programmed to perform any number of the functions as disclosed.

In the following, embodiments of the invention will be described in detail with reference to the accompanying drawings. It is to be understood that the following description of embodiments is not to be taken in a limiting sense. The scope of the invention is not intended to be limited by the embodiments described hereinafter or by the drawings, which are taken to be illustrative only.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

According to the techniques described herein, it is possible to manage medical data in a clinical workflow. In particular, it is possible to pseudonymize the medical data or at least parts thereof such as a patient identifier of the medical data where necessary. In particular, where medical data is stored outside of a local network, it is possible to implement the pseudonymization. On the other hand, medical data that remains within the local network may not be subject to the pseudonymization. According to various techniques described herein, it is possible to implement a backup mechanism where patient identifiers are persistently stored non-pseudonymized within the local network, e.g., of the hospital. A re-identification concept is possible: here, pseudonymized patient identifiers can be linked to non-pseudonymized patient identifiers, e.g., to reconstruct information. Thus, as will be appreciated from the above, pseudonymization may be implemented on an as-needed basis.

According to various scenarios, it would be possible that such functionality is implemented in a hospital picture archiving and communications system, e.g., for radiological images implementing the imaging data. This is sometimes referred to as Picture archiving and communication system (PACS). Here, medical data acquired may be push notified to multiple subscribers.

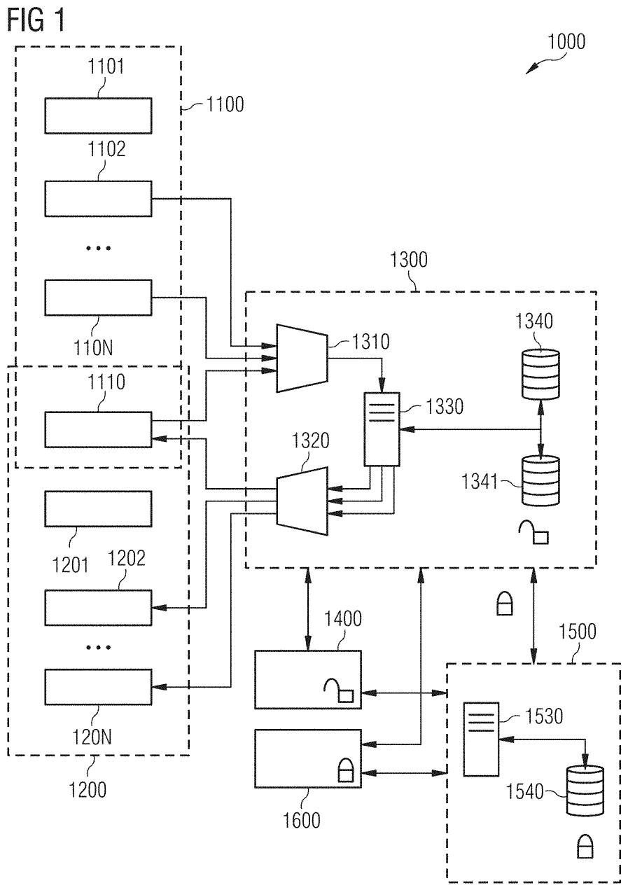

FIG. 1 schematically illustrates a medical data management system 1000 for managing medical data.

The medical data can generally comprise any digital data outputted by a measuring instrument designed to measure data which is medically relevant. Medical data can thus relate to imaging data such as X-rays and magnetic resonance scans, to electrocardiograms, measures of weight, temperature, blood pressure, etc.

Moreover, in order to allow such data to be associated to a specific patient, medical data can also comprise a patient identifier. The patient identifier can be any manner of a associating the medical data to a specific person, for instance a combination of name and surname, a social security number, an admission number in a hospital, etc.

Throughout the application reference is made to a non-pseudonymized patient identifier, or simply a patient identifier, and to a pseudonymized patient identifier. The non-pseudonymized patient identifier can allow a patient to be identified directly, for instance when the patient identifier is a combination or name and surname, or with a reduced effort, for instance when the patient identifier is a social security number or a patient number given by a hospital. In the latter cases, while a direct identification of the patient is not possible, the conversion of the patient identifier in a specific identity is rather easy since it only requires accessing the association between the value of the patient identifier and the identity, where this association might not be particularly difficult to retrieve. On the other hand, the pseudonymized patient identifier does not allow identifycation of a specific identity unless access is allowed to the key which was used to create the pseudonymized patient identifier based on the non-pseudonymized patient identifier. Since this key can be used only internally to the system 1300 and access to it can be severely limited and controlled, identification of the patient is prevented unless sufficient privacy rights are available.

The system 1000 can comprise a medical data gateway 1300, comprising a processor 1330 connectable to at least a plurality of input devices 1100. The input devices 1100 can be any measuring instrument designed to measure data which is medically relevant, such as those described above. The input devices 1100 are generally a plurality of input devices 1101, 1102-110N, 1110 from various manufacturers offering various data output standards. The medical data gateway 1300 is not specific to a given output standard and can operate with any digital medical data provided as input.

The medical data gateway 1300 can be connected to a local network. This is to be understood as being connected to a remote network, as will be discussed in the following. That is, the local network is not intended to limit the network in terms of size or capabilities. The local network can be a network of devices which have been given access rights higher than the devices in the remote network. In some cases, the local network could be, for instance, a network limited to a hospital, or a subset of the hospital network. The local network can be implemented in a wired and/or wireless manner with known technologies. The remote network could be, for instance, a network outside of the hospital, such as the internet, or a network in an insurance company, a service provider external to the hospital, etc.

That is, the medical data gateway 1300, which can be implemented as a PC or a server, can be connected to a network, which will be referred to a local network. This local network can be recognized from a remote network, in a manner per se known, for instance based on IP addresses. The local network and the remote network can be connected to each other in a manner per se known, so as to be able to exchange data. This allows nodes connected to the remote network to contact nodes in the local network, and vice versa. The possibility to distinguish the two networks allows a node in the local network to determine if the data, or connection request, comes from a node in the local network or from a node in the remote network.

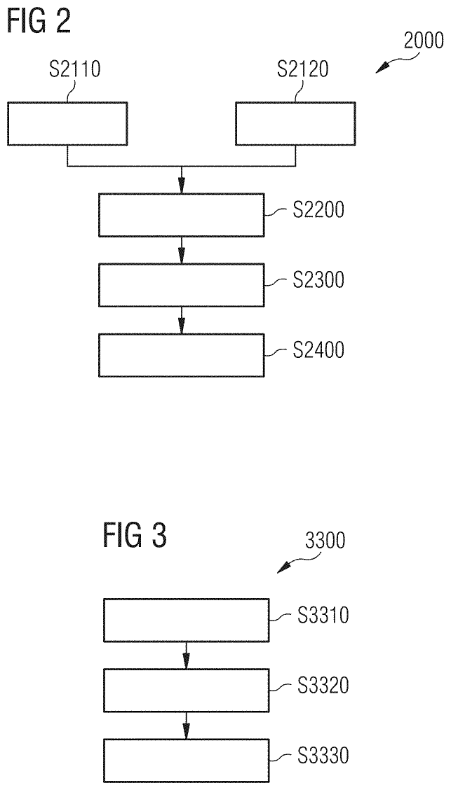

FIG. 2 schematically illustrates a method which can be implemented by the medical data gateway 1300. It will be clear that not all steps illustrated in FIG. 2 must be implemented in order for the method to be executed.

As can be seen in FIG. 2, the processor 1330 of the medical data gateway 1300 can be configured to carry out a step S2200 of collecting medical data, associated to a non-pseudonymized patient identifier and a data source identifier, from an input device 1101, 1102-110N, 1110 of the plurality of input devices 1100.

In some cases, the input device 1101, 1102-110N, 1110 is capable of providing the medical data already comprising a non-pseudonymized patient identifier. In some other cases, the non-pseudonymized patient identifier can be added to the medical data provided by the input device 1101, 1102-110N, 1110, for instance by the medical data gateway 1300 or by a device, or software, elaborating the data prior to forwarding them to the medical data gateway 1300.

As an example, the input device might be an X-ray machine collecting images of a patient. The patient identifier can be a patient name, or an ID number used by a hospital, which is entered by a medical operator when collecting the images. The patient identifier might be inputted, for instance, through a PC connected to the input device, or through the input device itself.

The data source identifier can be any field, for instance an alphanumeric string, capable of identifying one of the plurality of input devices 1100. This could be, for instance, a tag, a network address of the input device, etc.

Thanks to the collecting step S2200 it is thus possible to obtain medical data at the medical data gateway 1300. Once the medical data has been collected, the processor 1330 of the medical data gateway 1300 can be further configured to carry out a step S2300 of pseudonymizing at least the non-pseudonymized patient identifier of the medical data.

In general the pseudonymizing step S2300 can be implemented in any manner which allows the pseudonymized patient identifier not to reveal the identity of the patient. For instance, the non-pseudonymized patient identifier can be encrypted with a key, or a combination of the patient identifier and other parts of the medical data, such as the data source identifier, can be encrypted with a key. A hash value may be determined. Various algorithms for pseudonymizing data based on one or more secret keys are known to the skilled person and can be implemented. For instance, a private-public keying pair could be used. Here, the public key could be made generally available outside the local network; while the private key is preserved inside the local network.

Next, an example that can be used through the various embodiments disclosed herein is given in connection with the encryption:

Employing “AES 256” encryption on ‘M_{rd}’, which is medical data, e.g., a patient identifier, can be expressed as:

Cᵣd=fₑ(M_{rd},K)

Here, C_{rd} is cipher text of plain text and K is random key

Public Key:

PubtpKEK={e, n} where ‘e’ is d⁻¹ mod Φ(n) and integer ‘d’ is gcd(Φ(n), d)=1; 1<d<Φ(n)) and p/q are prime with n=p*q

Private Key:

PrivtpKEK={d, n} where ‘e’ is d⁻¹ mod Φ(n) and integer ‘d’ is gcd(Φ(n), d)=1; 1<d<Φ(n)) and p/q are prime with n=p*q

By applying RSA encryption on C_{rd},

one gets, C_{rd′}=C_{rd}^{e}(mod n) where C_{rd}<n

Decryption—i.e., implementing a retrieval of the non-pseudonymized patient identifier:

M_{rd}=M_{rd′}^{e}(mod n)

The above is a specific example and other encryption/decryption routines would be conceivable for the techniques described herein.

Thanks to the pseudonymizing step S2300 it is therefore possible to render at least the patient identifier of the medical data pseudonymized, thus allowing the medical data to be accessed while ensuring that the access is compliant with the privacy of the patient.

The processor 1330 of the medical data gateway can be further configured to carry out a step S2400 of exporting the pseudonymized medical data to a remote storage 1500. In preferred embodiments, the remote storage 1500 can be part of a remote network, external to the local network in which the medical data gateway 1300 runs.

In some embodiments, the remote storage 1500 can comprise at least a processor 1530 and a storage 1540. The processor can be configured to store the pseudonymized medical data on the storage 1540. In general, the remote storage 1500 can be implemented by a PC, a server, or similar data computing devices.

The processor 1330 of the medical data gateway 1300 can be further configured to carry out a step S2500 of allowing access S2500 to the non-pseudonymized patient identifier to a local application 1400, running in the local network, and/or refusing access to the non-pseudonymized patient identifier to a remote application 1600, running outside of the local network.

In some embodiments, this might be executed after a step of retrieving the medical data from remote storage 1500, though this is not mandatory. In particular, after retrieving the medical data, the user might need to identify the patient. This is the case, for instance, of a doctor, operating in the hospital and diagnosing a patient. In this case, by receiving a request from an application 1400 inside the local network, the medical data gateway can allow the correspondence between the pseudonymized patient identifier, received with the request from the local application 1400, and the non-pseudonymized patient identifier, to be accessed and return the pseudonymized patient identifier corresponding to the non-pseudonymized patient identifier which has been provided.

In other cases, in which the user of the remote application 1600 does not need, or is not allowed to, access the non-pseudonymized patient identifier, the medical data gateway can refuse access to the non-pseudonymized patient identifier.

In this manner it is advantageously possible for the medical data gateway to ensure that correct access to privacy sensitive non-pseudonymized patient identifiers is granted on the basis of whether the request is received from the local application 1400 or from the remote application 1600. It thus becomes possible to ensure privacy compliance by configuring the network in which the medical data gateway operates, namely the local network, to be accessible only to devices and/or applications associated with the right to access the non-pseudonymized patient identifier. Such network configuration can be implemented in several known manners, as will be clear to those skilled in the art. For instance, by only allowing access to the local network to devices which can be operated by personnel having the right to access the non-pseudonymized patient data. Such rights management of selected devices connected to a network can be implemented in manners per-se known.

This also advantageously allows an application to be designed only once and then operating both as local application 1400 and remote application 1600, thus saving development effort for the application. In particular, the operation of the application as local application 1400 and as remote application 1600 can be the same for the retrieval of the pseudonymized medical data from the remote storage 1500. The application can then show the medical data with the pseudonymized patient identifier. The application can then further be configured to request the access to the non-pseudonymized patient identifier, for instance based on an input from the user. Access control is advantageously managed by the medical data gateway 1300, so that no specific development has to be integrated in the application. The application can thus be designed to provide the non-pseudonymized patient identifier to the user only in case it is granted access from the medical data gateway.

The centralized approach in which the pseudonymized medical data is saved on the remote storage 1500 and the association between the pseudonymized patient identifier and the non-pseudonymized patient identifier is saved in the medical data gateway thus allows pseudonymized medical data to be easily accessed by applications running in the local and remote network, without specific design requirements for ensuring privacy of the data. It further allows those applications to be designed for collecting medical data from a single contact point with a single interface, namely the remote storage 1500, instead of dealing with the plurality of interfaces implemented by the various input devices 1100. Additionally it allows to control access to the privacy sensitive association between the pseudonymized patient identifier and the pseudonymized patient identifier in a simple manner based on a network configuration.

While in the description above the request to access the non-pseudonymized patient identifier has been described as being received by the medical data gateway 1300 directly from the local application 1400 or from the remote application 1600, the present invention is not limited to this implementation. In some cases the request for accessing the non-pseudonymized patient identifier can be received from the remote storage 1500, together with information indicating whether the request has originated from the local application 1400 or the remote application 1600. Still alternatively, or in addition, the various requests can be received by networks nodes such as routers, repeaters, etc. and forwarded to the intended destination node.

As visible in FIG. 1, the medical data gateway 1300 can further comprise a storage 1340, 1341. In some embodiments, the storage can be configured to comprise a storage 1340 and a backup storage 1341. Storage 1340 and 1341 can be implemented by memories, solid-state storage, hard-disk, locally implemented at the medical data gateway 1300 but also by remote storage accessible to the medical data gateway 1300.

FIG. 3 further illustrates a step S3300 of pseudonymizing which can be a possible further implementation of the pseudonymizing step S2300. In step S3300, as visible in FIG. 3, the processor 1330 of the medical data gateway 1300 can be further configured to carry out a step S3310 of creating a pseudonymized patient identifier based at least on the non-pseudonymized patient identifier and the data source identifier.

Moreover, in step S3300 the processor 1330 of the medical data gateway 1300 can be further configured to carry out a step S3320 of associating the medical data to the pseudonymized patient identifier. The association can be implemented for instance as a table, in which parts of the medical data, in particular at least the patient identifier, are associated with the pseudonymized patient identifier. Other known manners and data structures for associating data can be implemented.

Still further, in step S3300 the processor 1330 of the medical data gateway 1300 can be further configured to carry out a step S3330 of storing the pseudonymized patient identifier and the non-pseudonymized patient identifier in the storage 1340, 1341. It will be clear that this does not require the complete storage of the medical data in the storage 1340, 1341, which can thus be used efficiently. In general any storing manner which allows the non-pseudonymized patient identifier to be retrieved from the storage based on the respective pseudonymized patient identifier can be implemented.

In this manner it can be ensured that the privacy sensitive non-pseudonymized patient identifier, and its association to the pseudonymized patient identifier, is stored only once, in a controlled environment.

By further storing the pseudonymized medical data in the remote storage 1500, it can be ensured that no other copies of the medical data need to be kept, for instance in the input devices 1100. In particular, in some embodiments, it could be envisaged that, after the step of exporting S2400, all remaining copies of the medial data other than the copy on the remote storage 1500, are removed, for instance based on a command from the medical data gateway 1300.

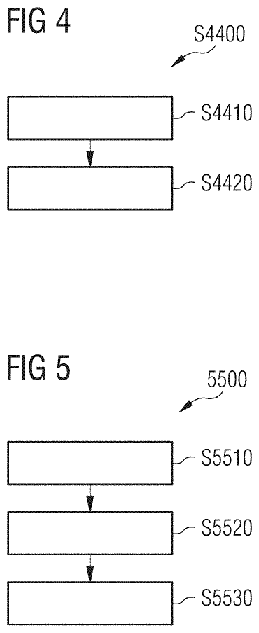

This is illustrated more in details in FIG. 4. In particular, FIG. 4 further illustrates a step S4400 of exporting which can be a possible further implementation of the exporting step S2400. In step S4400, as visible in FIG. 4, the processor 1330 of the medical data gateway 1300 can be further configured to carry out a step S4410 of transferring the pseudonymized medical data to the remote storage 1500, and a step S4420 of deleting the pseudonymized medical data from the medical data gateway 1300. In this case it will be clear that the non-pseudonymized patient identifier and its association to the pseudonymized patient identifier is not part of the deleted data.

FIG. 5 further illustrates a step S5500 of accessing which can be a possible further implementation of the accessing step S2500. In step S5500, as visible in FIG. 5, the processor 1330 of the medical data gateway 1300 can be further configured to carry out a step S5510 of receiving a conversion request comprising the pseudonymized patient identifier.

As discussed above, the request can be issued directly from the local application 1400 or from the remote application 1600. Alternatively, or in addition, the request can be issued from or through the remote storage 1500, indicating whether the origin of the request is the local application 1400 or the remote application 1600.

Additionally, the processor 1330 of the medical data gateway 1300 can be further configured to carry out a step S5520 of evaluating whether the conversion request has sufficient rights for obtaining the non-pseudonymized patient identifier. This can be implemented based, for instance, on the network address of the application. Based thereon it can be determined if the application is running in the local network, and has thus sufficient rights for accessing the non-pseudonymized patient identifier, or whether the application is running in the remote network and cannot therefore access the non-pseudonymized patient identifier.

In case of a positive result of the evaluating step S5520, thus indicating that the requesting application has sufficient rights for accessing the non-pseudonymized patient identifier, the processor 1330 of the medical data gateway 1300 can be further configured to carry out a step S5530 of converting the pseudonymized patient identifier into the corresponding non-pseudonymized patient identifier. The non-pseudonymized patient identifier can then be forwarded to the requesting application.

In the above a description has been provided concerning medical data which is sent to the medical data gateway 1300 from one of the input devices 1100. How precisely the data is transferred to the medical data gateway 1300 is a matter of implementation and several possible methods can be implemented. In the following some particularly advantageous manners will be described.

In some embodiments, as illustrated in FIG. 2, the processor 1330 can further be configured to carry out a step S2120 of receiving a data availability notification, comprising the data source identifier and the patient identifier. The data availability notification can be issued by the input device 1101-1102-110N corresponding to the indicated data source identifier. Thanks to this approach, the medical data gateway can be informed that medical data is available at a specific input device 1101, 1102-110N, 1110 for collection.

Alternatively, or in addition, the processor 1330 can further be configured to carry out a step S2110 of receiving a data query, comprising the data source identifier and the patient identifier. The data query can be issued, for instance, by the applications 1400, 1600, by the remote storage 1500 or by one of a plurality of output devices 1200. Thanks to this approach, data can be processed by the medical data gateway only when such data becomes relevant.

In both cases, the patient identifier could be the non-pseudonymized patient identifier or the pseudonymized patient identifier. In case the pseudonymized patient identifier is used, a conversion into the non-pseudonymized patient identifier might be needed in order to retrieve the data from the input devices 1100. In this case, if the data query or the availability notification came from an application or a device outside of the local network, the medical data gateway 1300 can be configured to refuse the conversion request from the pseudonymized patient identifier to the non-pseudonymized patient identifier as previously described. In this manner, possible security breaches in form of a data query or availability notification can be prevented.

In both cases, once the medical data gateway 1300 is informed of the data source identifier, identifying the input device from which the medical data is to be collected, a multiplexer 1310 can be operated so that the medical data can be transferred from the respective input device 1101, 1102-110N, 1110 to the processor.

In particular, in some embodiments, as illustrated in FIG. 1, in order to do so the system can further comprise a multiplexer 1310, configured to connect the processor 1330 to an input device 1101, 1102-110N, 1110 identified by the data source identifier. While the multiplexer 1310 is illustrated as being included in the medical data gateway 1300, the invention is not limited thereto and the multiplexer 1310 could alternatively be implemented outside of the medical data gateway 1300. Thanks to this approach, various input devices can be connected to a single medical data gateway 1300. It will be clear that the multiplexer 1310 can be implemented in hardware and/or in software.

In some embodiments, in order to increase safety of the medical data, the pseudonymizing step S2300, S3300 can be implemented each time medical data is received by one of the input devices 1101, 1102-110N, 1110. Particularly in cases in which the generation of the pseudonymized patient identifier is based not only on the non-pseudonymized patient identifier but also on other data which varies each time data is collected from one of the input devices 1101, 1102-110N, 1110, this could obtain a different pseudonymized patient identifier each time new data is retrieved from one of the input devices 1100. While this increases storage due to plurality of associations between the non-pseudonymized patient identifier and several pseudonymized patient identifiers, it increases the reliability of the privacy of the data. In particular, should one correspondence between a pseudonymized patient identifier and the respective non-pseudonymized patient identifier be identified by a hacker, or by mistake, this will only allow recognizing that the specific medical data associated to the given pseudonymized patient identifier corresponds to the respective non-pseudonymized patient identifier. Further medical data related to the same non-pseudonymized patient identifier will not be recognizable since it can be associated to a different pseudonymized patient identifier.

In some embodiments, as illustrated in FIG. 1, the system 1000 can further comprise a demultiplexer 1320 and/or a plurality of output devices 1200. It will be clear that the demultiplexer 1320 can be implemented in hardware and/or in software. The output devices can comprise output only devices, such as device 1201, 1202-120N, as well as input/output device such as device 1110. The output devices can be configured to output medical data retrieved through the medical data gateway 1300. In some embodiments, the medical data can be provided by the medical data gateway 1300 by retrieving this data from one or more of the input devices 1100. Alternatively, or in addition, the medical data can be provided by the medical data gateway 1300 by retrieving this data from the remote storage 1500.

The connection from the processor 1330 to the output devices 1200 can go through the demultiplexer 1320, when implemented. The demultiplexer 1320 can in particular allow different data flows, for instance associated to different protocols, or being redirected from one or more input devices 1100 as well as from the remote storage 1500, to the appropriate output device 1200.

From the description above it is therefore clear how the invention can allow or deny access, based on a network configuration, to non-pseudonymized patient identifiers. This simplifies the design and operation of applications operating on the medical data, which can be granted or refused access depending on their network connection instead of being designed in two different manners. Moreover, by storing the correspondence between the non-pseudonymized patient identifier and the pseudonymized patient identifier in a medical data gateway, control is maintained over the sensitive non-pseudonymized patient identifiers, thus increasing privacy compliancy and robustness of the system.

Summarising, above, techniques have been described which help to persistently store patient identifiers by providing a respective backup mechanism. The backup mechanism can restrict use of the patient identifiers within a local network, e.g., of a hospital. Pseudonymized patient identifiers can be uploaded to an external data storage. According to various techniques, a re-identification concept is provisioned where applications—e.g., medical applications assisting medical personnel—can fetch original patient identifiers when they are within the local network. This fetching can be based on the pseudonymized identifiers when the applications are executed outside of the local network.

While various embodiments have been described above, each comprising one or more features, it will be clear that the invention is not limited to the described embodiments in those specific forms. The skilled person will recognize that additional embodiments can be implemented by combining one or more feature from any of the embodiments with one or more features from any other embodiment.

For illustration, while various scenarios in the context of pseudonymized and non-pseudonymized data have been described in the context of medical data, similar techniques may also be applicable to other use cases, e.g., use cases that detect presence of people or activity of people, etc.

Of course, the embodiments of the method according to the invention and the imaging apparatus according to the invention described here should be understood as being example. Therefore, individual embodiments may be expanded by features of other embodiments. In particular, the sequence of the method steps of the method according to the invention should be understood as being example. The individual steps can also be performed in a different order or overlap partially or completely in terms of time.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A medical data management system for managing medical data, comprising:
a medical data gateway, comprising at least one processor connectable to a plurality of input devices, the medical data gateway being connected to a local network and the at least one processor being configured to carry out at least:
collecting medical data, associated to a non-pseudonymized patient identifier and a data source identifier, from an input device of the plurality of input devices,
pseudonymizing at least the non-pseudonymized patient identifier of the medical data to produce pseudonymized medical data,
exporting the pseudonymized medical data to a remote storage, the remote storage being part of a remote network, external to the local network, and
at least one of making the non-pseudonymized patient identifier accessible to a local application, running in the local network, and making the non-pseudonymized patient identifier non-accessible to a remote application, running outside of the local network.

2. The system of claim 1, wherein the medical data gateway further comprises:
a storage, and wherein, for the pseudonymizing, the at least one processor is further configured to carry out:
creating a pseudonymized patient identifier based on the non-pseudonymized patient identifier and the data source identifier,
associating the medical data to the pseudonymized patient identifier, and
storing the pseudonymized patient identifier and the non-pseudonymized patient identifier in the storage.

3. The system of claim 1, wherein, for the exporting, the at least one processor is further configured to carry out:
transferring the pseudonymized medical data to the remote storage, and
deleting the pseudonymized medical data from the medical data gateway.

4. The system of claim 1, wherein, for the accessing, the at least one processor is further configured to carry out:
receiving a conversion request including the pseudonymized patient identifier,
evaluating whether the conversion request has sufficient rights for obtaining the non-pseudonymized patient identifier, and
converting, upon the evaluating indicating that the conversion request has sufficient rights for obtaining the non-pseudonymized patient identifier, the pseudonymized patient identifier into the corresponding non-pseudonymized patient identifier.

5. The system of claim 1, wherein the at least one processor is further configured to carry out:
receiving a data availability notification, including the data source identifier and the patient identifier.

6. The system of claim 1, wherein the at least one processor is further configured to carry out:
receiving a data query, including the data source identifier and the patient identifier.

7. The system of claim 5, further comprising:
a multiplexer configured to connect the at least one processor to an input device identified by the data source identifier.

8. The system of claim 1, further comprising:
a demultiplexer, configured to connect the at least one processor to an output device.

9. A medical data management method for managing medical data, comprising:
collecting medical data, at a medical data gateway connected to a local network, associated to a non-pseudonymized patient identifier and a data source identifier, from an input device of a plurality of input devices;
pseudonymizing at least the non-pseudonymized patient identifier of the medical data to create pseudonymized medical data;
exporting the pseudonymized medical data to a remote storage, the remote storage being part of a remote network, external to the local network; and
at least one of making the non-pseudonymized patient identifier accessible to a local application, running in the local network, and making the non-pseudonymized patient identifier inaccessible to a remote application, running outside of the local network.

10. The method of claim 9, wherein the pseudonymizing comprises:
creating a pseudonymized patient identifier based on the non-pseudonymized patient identifier and the data source identifier;
associating the medical data to the pseudonymized patient identifier; and
storing the pseudonymized patient identifier and the non-pseudonymized patient identifier in a storage.

11. The method of claim 9, wherein the exporting comprises:
transferring the pseudonymized medical data to the remote storage; and
deleting the pseudonymized medical data from the medical data gateway.

12. The method of claim 9, wherein the accessing comprises:
receiving a conversion request including the pseudonymized patient identifier;
evaluating whether the conversion request has sufficient rights for obtaining the non-pseudonymized patient identifier; and
converting, upon the evaluating indicating that the conversion request has sufficient rights for obtaining the non-pseudonymized patient identifier, the pseudonymized patient identifier into the corresponding non-pseudonymized patient identifier.

13. The method of claim 9, further comprising:
receiving a data availability notification, including the data source identifier and the patient identifier.

14. The method of claim 9, further comprising:
receiving a data query, including the data source identifier and the patient identifier.

15. The method of claim 13, further comprising:
multiplexing a connection between at least one processor of the medical data gateway and an input device identified by the data source identifier.

16. The method of claim 9, further comprising:
demultiplexing a connection between at least one processor of the medical data gateway and an output device.

17. The system of claim 2, wherein, for the exporting, the at least one processor is further configured to carry out:
transferring the pseudonymized medical data to the remote storage, and
deleting the pseudonymized medical data from the medical data gateway.

18. The system of claim 2, wherein, for the accessing, the at least one processor is further configured to carry out:
receiving a conversion request including the pseudonymized patient identifier,
evaluating whether the conversion request has sufficient rights for obtaining the non-pseudonymized patient identifier, and
converting, upon the evaluating indicating that the conversion request has sufficient rights for obtaining the non-pseudonymized patient identifier, the pseudonymized patient identifier into the corresponding non-pseudonymized patient identifier.

19. The system of claim 6, further comprising:
a multiplexer configured to connect the at least one processor to an input device identified by the data source identifier.

20. The system of claim 7, further comprising:
a demultiplexer, configured to connect the at least one processor to an output device.

21. The system of claim 19, further comprising:
a demultiplexer, configured to connect the at least one processor to an output device.

22. The method of claim 10, wherein the exporting comprises:
transferring the pseudonymized medical data to the remote storage; and
deleting the pseudonymized medical data from the medical data gateway.

23. The method of claim 10, wherein the accessing comprises:
receiving a conversion request including the pseudonymized patient identifier;
evaluating whether the conversion request has sufficient rights for obtaining the non-pseudonymized patient identifier; and
converting, upon the evaluating indicating that the conversion request has sufficient rights for obtaining the non-pseudonymized patient identifier, the pseudonymized patient identifier into the corresponding non-pseudonymized patient identifier.

24. The method of claim 14, further comprising:
multiplexing a connection between at least one processor of the medical data gateway and an input device identified by the data source identifier.

25. The method of claim 15, further comprising:
demultiplexing a connection between the at least one processor of the medical data gateway and an output device.

26. The method of claim 24, further comprising:
demultiplexing a connection between the at least one processor of the medical data gateway and an output device.

27. A non-transitory computer-readable storage medium storing program code, loadable and executable by at least one processor, which when loaded and executed by the at least one processor, enabling the at least one processor to perform a method for managing medical data, comprising:
collecting medical data, at a medical data gateway connected to a local network, associated to a non-pseudonymized patient identifier and a data source identifier, from an input device of a plurality of input devices;
pseudonymizing at least the non-pseudonymized patient identifier of the medical data to create pseudonymized medical data;
exporting the pseudonymized medical data to a remote storage, the remote storage being part of a remote network, external to the local network; and
at least one of making the non-pseudonymized patient identifier accessible to a local application, running in the local network, and making the non-pseudonymized patient identifier inaccessible to a remote application, running outside of the local network.
